(19) United States
(12) Patent Application Publication
(10) Pub. No.: US 2011/0056003 A1
(43) Pub. Date: Mar. 10, 2011

(54) STRUCTURE OF SWIMMING GOGGLES

(76) Inventor: YING-RU CHEN, Xinzhuang City (TW)

(21) Appl. No.: 12/554,001

(22) Filed: Sep. 4, 2009

(51) Int. Cl. A61F 9/02

(52) U.S. Cl. 2/440; 2/452

(57) ABSTRACT

A structure of swimming goggles includes a pair of flexible gaskets, a pair of flexible lenses, a pair of frame clamps, and a pair of buckles. The flexible gaskets are integrally connected to each other by a connected section arranged therebetween. The flexible lenses are respectively fit to and received in the first and second flexible gaskets. The first and second frame clamps are respectively fit over the first and second flexible gaskets. The buckles are respectively mounted to openings defined in the frame clamps. A uses may change lenses of different light transmittances based on his or her needs and the resilient restoration of the frames and the lenses, after being bent, provides excellent water tightness therebetween so that difficulty of assembling and manufacturing costs of the lenses can be reduced.

### (a) TECHNICAL FIELD OF THE INVENTION

The present invention generally relates to a structure of swimming goggles, and more particularly to structure of swimming goggles that is formed by integration of flexible frames and lenses that are then clamped together by rigid frame clamps.

### (b) DESCRIPTION OF THE PRIOR ART

Swimming is a leisure activity that improves lung capacity and exercises the muscles of whole body and provides excellent help to improve body health. Due to the buoyancy of water, when a person is swimming, his or her body is supported by the floating power of water so that the gravitational burden applied to the body is reduced so that swimming particularly suit persons that are doing rehabilitation. However, human eyes may get soar and vision blurred in the water so that human cannot stay long in the water and must be assisted by using swimming goggles to enjoy the pleasure of swimming and diving.

An example of a conventional structure of swimming goggles is shown in FIG. 1 of the attached drawings, generally designated at 10. The swimming goggles 10 comprise a mask 11, two lenses 12, and a head strap 13. The mask 11 comprises two frames 111 to which the lenses 12 are respectively fit for providing tight engagement with a user's face when the user wears the goggles. The lenses are integrally formed and are made of polycarbonate. Each lens 12 has a front flat section 121 and a skirt 122 extending circumferentially around the flat section 121 so that an interior space is defined in the lens 12. The head strap 13 is connected between opposite ends of the mask 11. The head strap 13 is made of resilient silicone material to be fit over the head of the user for adjusting the length and tightness to fit the user's head.

The lenses 12 of the conventional swimming goggles 10 are made of polycarbonate and the lenses 12 must be manufactured with excellent flatness of the front flat sections. Further, the material of polycarbonate may generate internal stress therein, which is caused by cold shrink of large molecular chains during the fusion and process of the plastic material. If the plastic product cannot bear the internal stress, then crack or deformation may be formed. Thus, the process must be carried out carefully and this certainly raises the difficulty of producing process. Further, the mask 11 and the lenses 12 are mounted together by application of adhesives therebetween, which may easily deteriorate in quality thereof and thus leading to damage, and which also raises difficulty in assembling of parts.

In view of the drawbacks of the conventional swimming goggles 10 occurring in the manufacturing and assembling thereof, the present invention aims to provide a novel structure of swimming goggles that first combines flexible gaskets and lenses, and then fits rigid frame clamps to peripheral of the gaskets to complete the assembling. Swimming goggles with flexible lenses are a completely new product, which eliminates or alleviates the generation of internal stress and also improves the convenience of assembling.

### SUMMARY OF THE INVENTION

An objective of the present invention is to provide a structure of swimming goggles to facilitate the convenience of assembling so as to reduce the difficulty of assembling and defective rate of assembling.

Another objective of the present invention is to provide a structure of swimming goggles that reduces manufacturing costs of processing lenses of the goggles.

To achieve the above objectives, the present invention provide a structure of swimming goggles, which comprises first and second flexible gaskets, which are connected to each other by a coupling section arranged therebetween, the first and second flexible gaskets having inner circumferences respectively defining first and second grooves; first and second flexible lenses, which are shaped to respectively correspond to the first and second grooves for being fit to and received in the first and second flexible gaskets; first and second frame clamps respectively corresponding to and fit over the first and second flexible gaskets and respectively forming first and second openings at the sides of the first and second gaskets that are opposite to the coupling section; and first and second buckles, which respectively mounted to and positioned on the first and second openings of the first and second frame clamps to secure the first and second frame clamps to the first and second flexible gaskets.

In an embodiment, a head strap is inserted between the first and second buckles, and the opposite ends of head strap are respectively inserted to both side of the first and second buckles. The first and second flexible gaskets and the connected section can be integrally formed together as one-piece, and the first and second flexible gaskets and the connected section are made of rubber material.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of a conventional structure of swimming goggles.

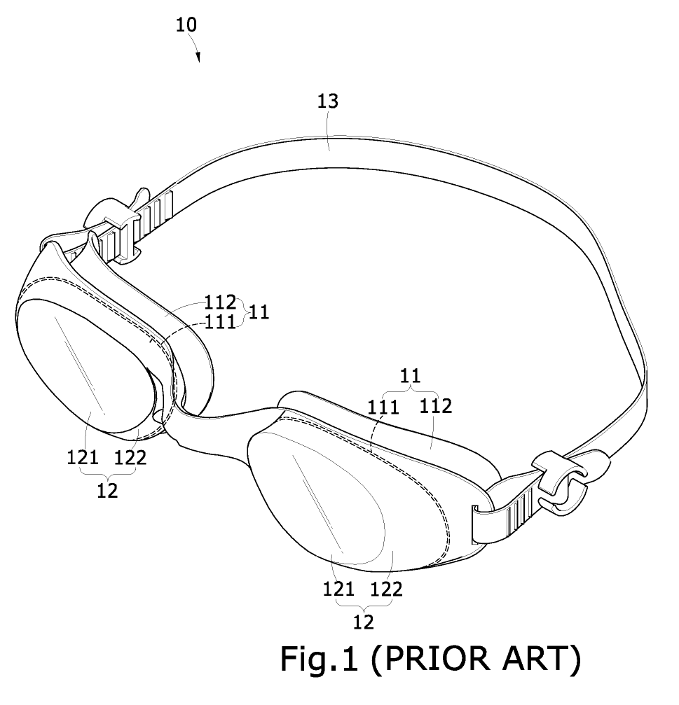

FIG. 2 is an exploded view of swimming goggles in accordance with the present invention.

FIG. 3 is another exploded view of the swimming goggles in accordance with the present invention.

FIG. 4 is a perspective view of the swimming goggles in accordance with the present invention.

FIG. 5 is a cross-sectional view taken along line A-A of FIG. 4.

FIG. 6 is a cross-sectional view taken along line B-B of FIG. 4.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

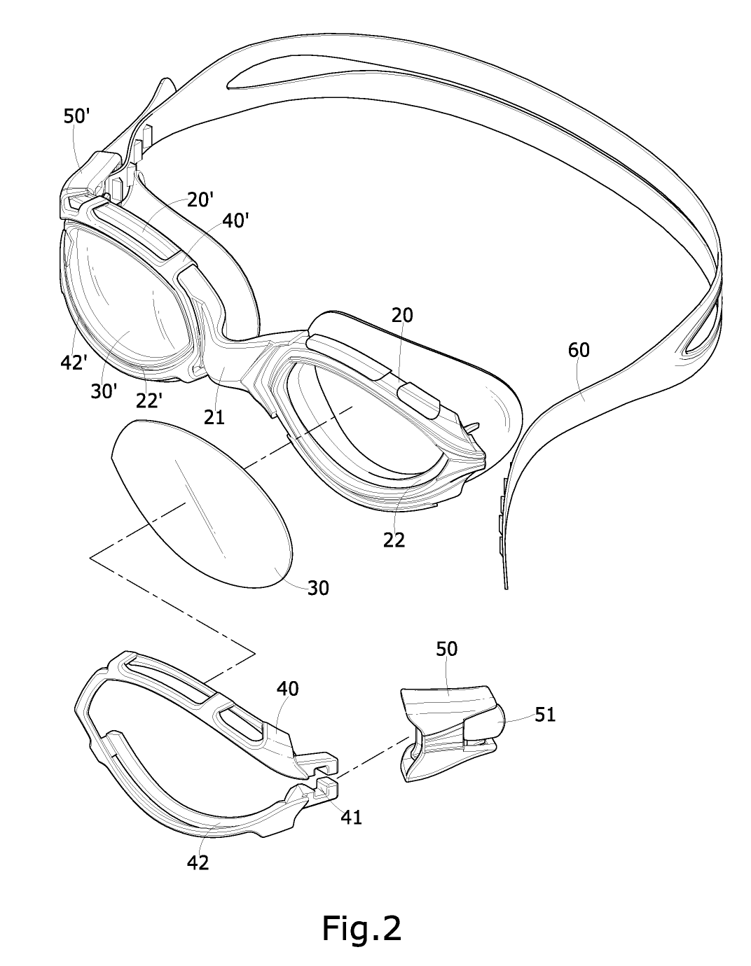

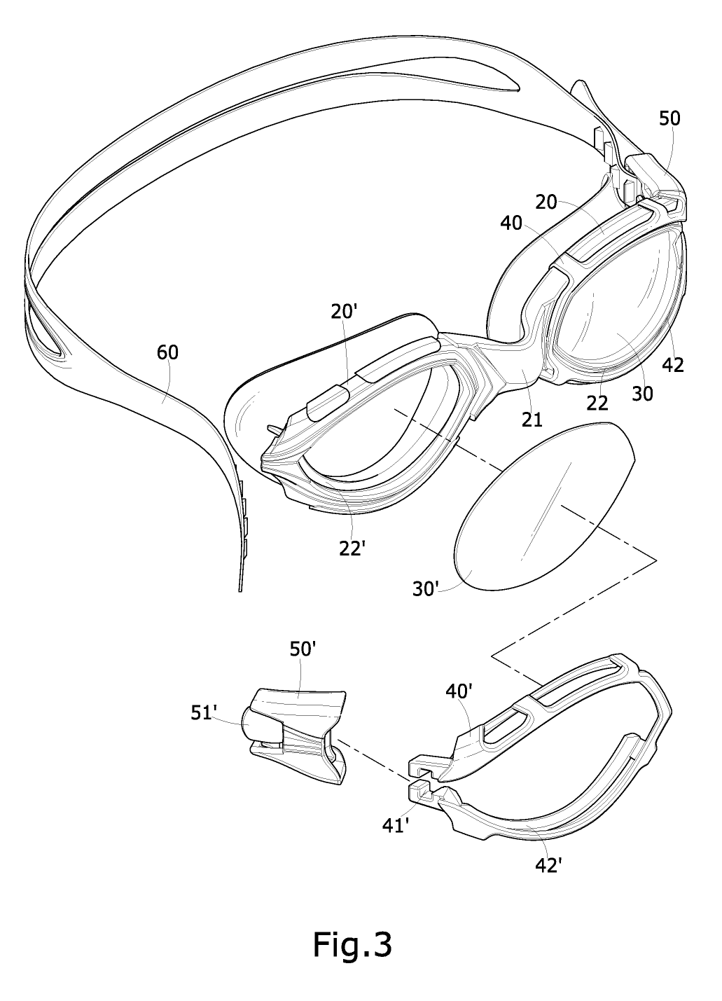

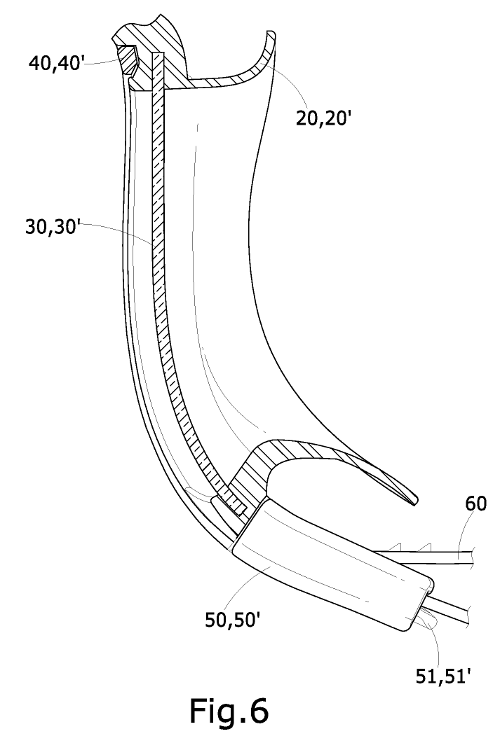

Referring to FIGS. 2-6, exploded views, a perspective view in an assembled form, and cross-sectional views of a structure of swimming goggles in accordance with the present invention are shown. The structure of the swimming goggles in accordance with the present invention will be described in details as follows.

The structure of swimming goggles comprises a first flexible gasket 20 and a second flexible gasket 20′. The first and second flexible gaskets 20, 20′ are respectively and integrally formed with elastic materials. The first and second flexible gaskets 20, 20′ are connected to each other by a connected section 21. As shown in the drawings, it is preferable that the first and second flexible gaskets 20, 20′ and the connected section 21 are integrally formed as a elastic gasket. Consequently, the first flexible gasket 20 and the second flexible gasket 20′, and the connected section 21 all have the nature of resilient flexibility and may undergo change of shape according to the condition of practical uses so as to more closely suit the needs of a user. Further, the first flexible gasket 20 and the second flexible gasket 20′ have inner circumferences respectively defining a first groove 22 and a second groove 22′.

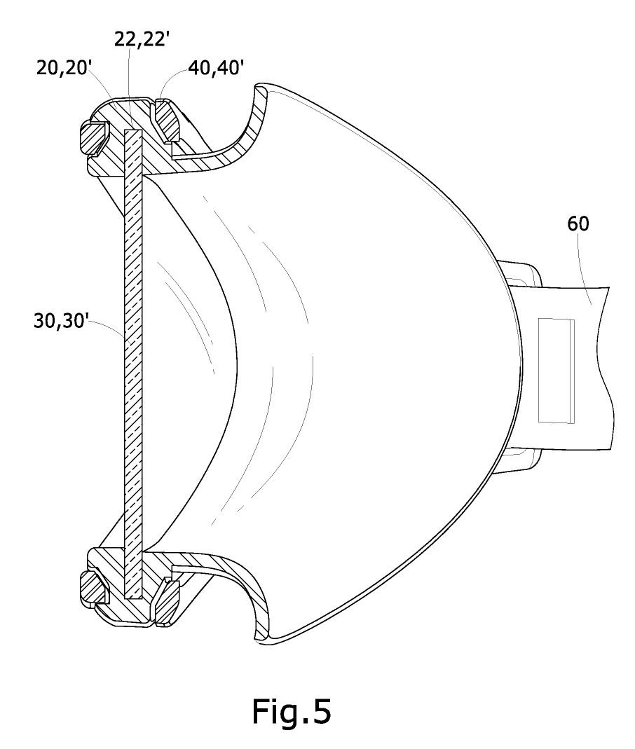

A first flexible lens 30 and a second flexible lens 30′ are shaped to respectively correspond to the first groove 22 and the second groove 22′ for being fit to and received in the first flexible gasket 20 and the second flexible gasket 20′. Further, as shown in FIG. 5, when the first flexible lens 30 and the second flexible lens 30′ are respectively fit into the first groove 22 and the second groove 22′, due to the resilient flexibility of the flexible lenses 30, 30′, the curvatures thereof will be changed to fit the shapes of the flexible gaskets 20, 20′ thereby enhancing the tightness of engagement of the fitting.

A first frame clamp 40 and a second frame clamp 40′ are respectively and integrally formed with rigid plastic materials. As shown in FIG. 5, the frame clamps 40, 40′ show an inverted U-shaped in the cross-sections thereof. To assemble, the frame clamps 40, 40′ are respectively corresponding to and fit over outside the first flexible gasket 20 and the second flexible gasket 20′. With the materials that make the frame clamps 40, 40′ being rigid materials, the frame clamps 40, 40′ provide clamping forces to clamp the flexible gaskets 20, 20′ and the flexible lenses 30, 30′ together thereby further enhancing the tightness of engagement between the flexible gaskets 20, 20′ and the flexible lenses 30, 30′. Further, the first frame clamp 40 and the second frame clamp 40′ respectively form a first opening 41 and a second opening 41′ at the sides of the first flexible gasket 20 and the second flexible gasket 20′ that are opposite to the connected section 21 and also form inward bent sections 42, 42′ respectively adjacent to the first opening 41 and the second opening 41′ to ensure closer compliance with facial contour of the user.

A first buckle 50 and a second buckle 50′ are respectively mounted to and positioned on the first opening 41 and the second opening 41′ of the first frame clamp 40 and the second frame clamp 40′ to secure the first frame clamp 40 and the second frame clamp 40′ outside the first flexible gasket 20 and the second flexible gasket 20′ respectively and to lock the first opening 41 of the first frame clamp 40 and the second opening 41′ of the second frame clamp 40′ thereby completely close the first opening 41 of the first frame clamp 40 and the second opening 41′ of the second frame clamp 40′ for preventing undesired separation from occurring in the use of the swimming goggles. Further, a head strap 60 is arranged between the first buckle 50 and the second buckle 50′. The head strap 60 has opposite ends respectively attached to the first buckle 50 and the second buckle 50′. The first buckle 50 and the second buckle 50′ are further and respectively provided with a first hold-down button 51 and the second hold-down button 51′ to clamp and secure the head strap 60 in such a way to facilitate adjustment of tautness of the head strap 60 by simply depressing and releasing the buttons.

To conclude, to assemble the present invention, the lenses and the gaskets that are flexible are first fit to each other to form semi-finished sub-assemblies and then the rigid frame clamps are used to tightly fix the semi-finished sub-assemblies. In the course of fitting and clamping, the flexible lenses 30, 30′ and the flexible gaskets 20, 20′ may undergo deformation due to the compression by the frame clamps 40, 40′ so as to realize tight engagement between the first flexible lens 30 and the first flexible gasket 20 and also elimination of adhesive application and adjustment carried out by operators. Thus, the convenience of assembling is enhanced and the defective rate of product assembling is reduced. Further, the manufacturing of the flexible lenses 30, 30′ does not need processing for three-dimensional configuring so that the expense of manufacturing is reduced.

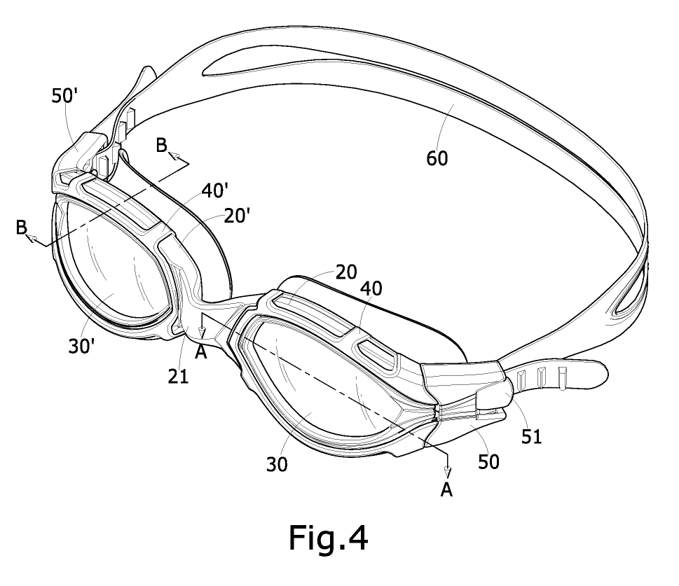

Referring to FIGS. 4-6, when the first frame clamp 40 is fit over and clamps the first flexible gasket 20 and the second frame clamp 40′ is fit over and clamps the second flexible gasket 20′, the clamping forces induced by the frame clamps 40, 40′ to clamp the flexible gaskets 20, 20′ may cause deformation of the flexible gaskets 20, 20′ and the flexible lenses 30, 30′, so that, as shown in the drawings, the flexible gaskets 20, 20′, as being clamped by the frame clamps 40, 40′, may be forcibly fit into and secured in the interior spaces defined by the frame clamps 40, 40′, making the flexible gaskets 20, 20′ more tightly enclosing the flexible lenses 30, 30′. Further, the flexible lenses are also deformed by being clamped by the frame clamps 40, 40′ so that due to the nature of the materials of the flexible lenses 30, 30′, the flexible lenses 30, 30′ may change the surface curvatures thereof by following the contours of the bent sections 42, 42′. Thus, additional processing of the lens can be eliminated.

As described above and shown in FIGS. 2-6, in practice, the present invention offers the following advantages:

(1) The present invention uses the deformation that flexible gaskets and lenses undergo when they are clamped by rigid frame clamps to improve the convenience of assembling and uses the resilient restoration of the lenses to improve tightness of engagement in the assembling operation.

(2) Further, the flexible lenses 30, 30′ of the present invention may automatically deform to comply with the shapes of the frame clamps 40, 40′ so that additional processing is not needed and the manufacturing costs are reduced.

However, it is understood that the above describes a preferred embodiment and does not intend to limit the scope of the present invention. Modifications and substitutions, such as change of the shapes, sizes, and materials of the flexible gaskets 20, 20′ and the flexible lenses 30, 30′, or change of the shape, size, and material of the frame clamps 40, 40′, or change of the structure of the buckles 50, 50′, are considered belonging to the scope of the present invention. Thus, equivalent or easy modifications that can be made by those having ordinary skills in the art without departing from the spirits and scope of the present invention are covered by the scope of the appended claims.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A structure of swimming goggles, comprising:
first and second flexible gaskets, the first and second flexible gaskets being connected to each other by a connected section arranged therebetween, the first and second flexible gaskets having inner circumferences respectively defining first and second grooves;
first and second flexible lenses shaped to respectively correspond to the first and second grooves for being fit to and received in the first and second flexible gaskets;
first and second frame clamps respectively corresponding to and fit over the first and second flexible gaskets and respectively forming first and second openings at sides of the first and second gaskets that are opposite to the connected section; and
first and second buckles respectively mounted to and positioned on the first and second openings of the first and second frame clamps to secure the first and second frame clamps to the first and second flexible gaskets.

2. The structure of swimming goggles according to claim 1 further comprising a head strap arranged between the first and second buckles, the head strap having opposite ends respectively attached to the first and second buckles.

3. The structure of swimming goggles according to claim 1, wherein the first and second flexible gaskets and the connected section are integrally formed together.

4. The structure of swimming goggles according to claim 3, wherein the first and second flexible gaskets and the connected section are integrally formed as a elastic gasket.
